(19) United States
(12) Patent Application Publication
(10) Pub. No.: US 2008/0268442 A1
(43) Pub. Date: Oct. 30, 2008

(54) METHOD AND SYSTEM FOR PREPARING A BLOOD SAMPLE FOR A DISEASE ASSOCIATION GENE TRANSCRIPT TEST

(75) Inventor: Dean Iverson Sproles, Seattle, WA (US)

Correspondence Address: DAVIS WRIGHT TREMAINE, LLP/Seattle, 1201 Third Avenue, Suite 2200, SEATTLE, WA, 98101-3045, US

(73) Assignee: IGD INTEL, LLC, Seattle, WA (US)

(21) Appl. No.: 11/750,538

(22) Filed: May 18, 2007

Related U.S. Application Data
(60) Provisional application No. 60/913,755, filed on Apr. 24, 2007.

(51) Int. Cl. C12Q 1/68

(52) U.S. Cl. 435/6

(57) ABSTRACT

System and method for preparing a blood sample for a disease association gene transcript test. Disease considerations for this unique test include a custom set of genetic sequences associated in peer-reviewed literature with various known diseases such as Addison's disease, anemia, asthma, atherosclerosis, autism, breast cancer, estrogen metabolism, Grave's disease, hormone replacement therapy, major histocompatibility complex (MHC) genes, longevity, lupus, multiple sclerosis, obesity, osteoarthritis, prostate cancer, and type 2 diabetes. The base dataset may be developed through clinical samples obtained by third-parties. Online access of real-time phenotype/genotype associative testing for physicians and patients may be promoted through a testing service.

### CROSS-REFERENCE TO PROVISIONAL PATENT APPLICATION

This patent application claims priority from a related provisional patent application entitled ‘BROAD-BASED DISEASE ASSOCIATION GENE TRANSCRIPT TEST’ filed on Apr. 24, 2007 which is incorporated herein in its entirety.

### BACKGROUND

Genetic diseases afflict many people and remain the subject of much study and misunderstanding. Some genetic disorders may be caused by the abnormal chromosome number, as in Down syndrome (extra chromosome 21) and Klinefelter's syndrome (a male with 2× chromosomes). Triplet expansion repeat mutations can cause fragile X syndrome or Huntington's disease, by modification of gene expression or gain of function, respectively. Other genetic disorders occur when specific gene sequences are not maintained as expected, such as with Multiple Sclerosis and Type II diabetes. Currently, around 4,000 genetic disorders are known, with more being discovered as more is understood about the human genome. Most disorders are quite rare and affect one person in every several thousands or millions while other are more common such as cystic fibrosis wherein about 5% of the population of the United States carry at least one copy of the defective gene.

A person's genetic makeup is reflected through Deoxyribonucleic Acids (DNA). DNA is a molecule that is comprised of sequences of nucleic acids that form the code which contains the genetic instructions for the development and functioning of living organisms. A DNA sequence or genetic sequence is a succession of any of four specific nucleic acids representing the primary structure of a real or hypothetical DNA molecule or strand, with the capacity to carry information. As is well understood in the art, the possible nucleic acids (letters) are A, C, G, and T, representing the four nucleotide subunits of a DNA strand-adenine, cytosine, guanine, and thymine bases covalently linked to phospho-backbone. Typically the sequences are printed abutting one another without gaps, as in the sequence AAAGTCTGAC. A succession of any number of nucleotides greater than four may be called a sequence. With regard to its biological function, which may depend on context, a sequence may be sense or anti-sense, and either coding or non-coding.

Ribonucleic acid (RNA) is a nucleic acid polymer consisting of nucleotide monomers, that acts as a messenger between DNA and ribosomes, and that is also responsible for making proteins by coding for amino acids. RNA polynucleotides contain ribose sugars unlike DNA, which contains deoxyribose; and RNA substitutes a uracil in any position of DNA where thymine is present. RNA is transcribed (synthesized) from DNA by enzymes called RNA polymerases and further processed by other enzymes. RNA serves as the template for translation of genes into proteins, transferring amino acids to the ribosome to form proteins, and also translating the transcript into proteins.

A gene is a segment of nucleic acid that contains the information necessary to produce a functional product, usually a protein. Genes contain regulatory regions dictating under what conditions the product is produced, transcribed regions dictating the structure of the product, and/or other functional sequence regions. Genes interact with each other to influence physical development and behavior. Genes consist of a long strand of DNA (RNA in some viruses) that contains a promoter, which controls the activity of a gene, and a coding sequence, which determines what the gene produces. When a gene is active, the coding sequence is copied in a process called transcription, producing an RNA copy of the gene's information. This RNA can then direct the synthesis of proteins via the genetic code. However, RNA can also be used directly, for example as part of the ribosome. These molecules resulting from gene expression, whether RNA or protein, are known as gene products.

The total complement of genes in an organism or cell is known as its genome. The genome size of an organism is loosely dependent on its complexity. The number of genes in the human genome is estimated to be just under 3 billion base pairs and about 30,000 genes.

As previously mentioned, certain genetic disorders may result from DNA sequences being incorrectly coded. A Single Nucleotide Polymorphism or S.N.P. (often time called a “snip”) is a DNA sequence variation occurring when a single nucleotide—A, T, C, or G—in the genome (or other shared sequence) differs between members of a species (or between paired chromosomes in an individual). For example, two sequenced DNA fragments from different individuals, MGCCTA to MGCTTA, contain a difference in a single nucleotide. In this case we say that there are two alleles: C and T. High degrees of variation within coding and non-coding regions exist and are the topic of ongoing research efforts.

Within a population, Single Nucleotide Polymorphisms can be assigned a minor allele frequency—the ratio of chromosomes in the population carrying the less common variant to those with the more common variant. Usually one will want to refer to Single Nucleotide Polymorphisms with a minor allele frequency of >1% (or 0.5% etc.), rather than to “all Single Nucleotide Polymorphisms” (a set so large as to be unwieldy). It is important to note that there are variations between human populations, so a Single Nucleotide Polymorphism that is common enough for inclusion in one geographical or ethnic group may be much rarer in another.

Single Nucleotide Polymorphisms may fall within coding sequences of genes, noncoding regions of genes, or in the intergenic regions between genes. Single Nucleotide Polymorphisms within a coding sequence will not necessarily change the amino acid sequence of the protein that is produced, due to degeneracy of the genetic code. A Single Nucleotide Polymorphism in which both forms lead to the same polypeptide sequence is termed synonymous (sometimes called a silent mutation)—if a different polypeptide sequence is produced they are non-synonymous. Single Nucleotide Polymorphisms that are not in protein coding regions may still have consequences for gene splicing, transcription factor binding, or the sequence of non-coding RNA.

Variations in the DNA sequences of humans can affect how humans develop diseases, and/or respond to pathogens, chemicals, drugs, etc. However, one aspect of learning about DNA sequences that is of great importance in biomedical research is comparing regions of the genome between people (e.g., comparing DNA sequences from similar people, one with a disease and one without the disease). Technologies from Affymetrix™ and Illumina™ (for example) allow for genotyping hundreds of thousands of Single Nucleotide Polymorphisms for typically under $1,000.00 in a couple of days.

A microarray (also commonly known as gene or genome chip, DNA chip, or gene array) is a collection of microscopic DNA wells or probes, commonly representing single genes, arrayed on a solid surface by covalent attachment to chemically suitable matrices. DNA arrays are different from other types of microarray only in that they either measure DNA or use DNA as part of its detection system. Qualitative and quantitative measurements with DNA microarrays utilize the selective nature of DNA-DNA or DNA-RNA hybridization under high-stringency conditions and fluorophore-based detection.

Microarrays are commonly used for expression profiling, i.e., monitoring expression levels of thousands of genes simultaneously, or for comparative genomic hybridization. Microarray analysis techniques are typically used in interpreting the data generated from experiments on DNA, RNA, and protein microarrays, which allow researchers to investigate the expression state of a large number of genes—in many cases, an organism's entire genome—in a single experiment. Such experiments generate a very large volume of genetic data that can be difficult to analyze, especially in the absence of good gene annotation. Most microarray manufacturers, such as Affymetrix™, provide commercial data analysis software with microarray equipment such as plate readers.

Specialized software tools for statistical analysis to determine the extent of over- or under-expression of a gene in a microarray experiment relative to a reference state have also been developed to aid in identifying genes or gene sets associated with particular phenotypes. Such statistics packages typically offer the user information on the genes or gene sets of interest, including links to entries in databases such as NCBI's GenBank and curated databases such as Biocarta and Gene Ontology.

As a result, genotyping refers to the process of determining the genotype of an individual with a biological assay. Current methods of doing this include Polymerase Chain Reaction (PCR), DNA sequencing, and hybridization to DNA microarrays or beads. The technology is intrinsic for test on father-/motherhood and in clinical research for the investigation of disease associated genes.

Further, phenotyping is also a known process for assessing phenotypes. The phenotype of an individual organism is either its total physical appearance and constitution or a specific manifestation of a trait, such as size, eye color, or behavior that varies between individuals. Phenotype is determined to a large extent by genotype, or by the identity of the alleles that an individual carries at one or more positions on the chromosomes. Many phenotypes are determined by multiple genes and influenced by environmental factors. Thus, the identity of one or a few known alleles does not always enable prediction of the phenotype.

In a drawback of the current state of the art, this genotyping process is typically accomplished for a single patient or research sample in a single sampling for a single iteration and with a specific disease in mind for the genotyping. As such, the results are relatively isolated with respect to any possible comparison and analysis of other similarly situated patients. Furthermore, such isolation leads to inefficiencies in diagnostics and treatment of the underlying results of the test. Without a system for allowing the sharing of underlying data, all potential benefits of aggregating the data are lost. Thus, as blood samples are collected, they are done so from an individualistic approach without regard for benefits to be realized from aggregating the data from may blood samples from many sample sources (i.e., people). What is needed is a broad-based disease association gene transcript test capable of allowing the assimilation of a wide range of data from a wide range of sources.

### BRIEF DESCRIPTION OF THE DRAWINGS

The foregoing aspects and many of the attendant advantages of the claims will become more readily appreciated as the same become better understood by reference to the following detailed description, when taken in conjunction with the accompanying drawings, wherein:

FIG. 1 shows a diagram of a method for preparing a blood sample and a microarray to be used in a broad-based disease association gene transcript test according to an embodiment of an invention disclosed herein;

FIG. 2 shows a diagrammatic representation of a method for collecting blood samples from several sources and isolating strands of genetic material for grouping according to an embodiment of an invention disclosed herein;

FIG. 3 shows a typical arrangement of the results of a diagnostic obtained from genetic material from blood samples in a microarray suitable for analysis in a broad-based disease association gene transcript test according to an embodiment of an invention disclosed herein; and

FIG. 4 shows a typical arrangement of data that may be associated in a database of information derived from a broad-based disease association gene transcript test according to an embodiment of an invention disclosed herein.

### DETAILED DESCRIPTION

The following discussion is presented to enable a person skilled in the art to make and use the subject matter disclosed herein. The general principles described herein may be applied to embodiments and applications other than those detailed above without departing from the spirit and scope of the present detailed description. The present disclosure is not intended to be limited to the embodiments shown, but is to be accorded the widest scope consistent with the principles and features disclosed or suggested herein.

The subject matter disclosed herein is related to transcriptional detection of single nucleotide polymorphisms (SNP) and insertion/deletion (I/D) genetic polymorphisms through a proportional analysis of RNA sequences detected through fluorescence hybridization on a custom manufactured microarray gene expression platform. SNPs may be identified through a specific design method (SNPs are typically assessed through DNA analysis). Disease considerations for this unique test include a custom set of genetic sequences associated in peer-reviewed literature with various known diseases such as Addison's disease, anemia, asthma, atherosclerosis, autism, breast cancer, estrogen metabolism, Grave's disease, hormone replacement therapy, major histocompatibility complex (MHC) genes, infectious disease screening panel, longevity, lupus, multiple sclerosis, obesity, osteoarthritis, prostate cancer, and type 2 diabetes. The base dataset may be developed through clinical samples obtained by third-parties clinical groups, and in partial association with the Swank M S Foundation. Further, coordination and volunteer efforts from followers of the Swank Program, as defined in the Multiple Sclerosis Diet Book (authored by Roy L Swank) may be assimilated and utilized. Online access of real-time phenotype/genotype associative testing for physicians and patients may be promoted through a testing service.

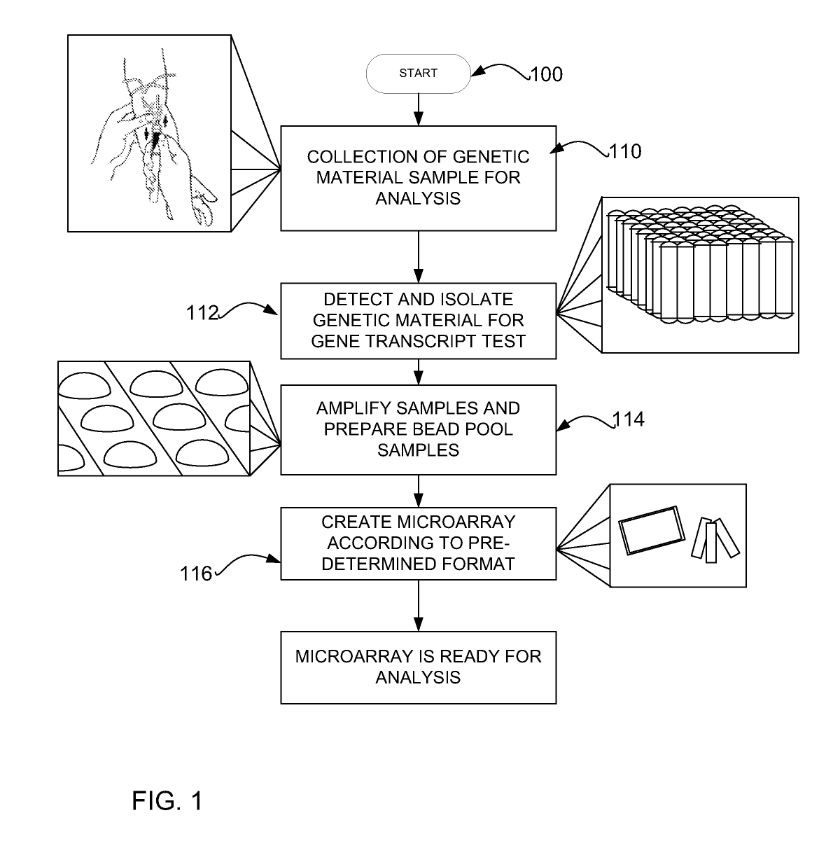

Various embodiments and methods of new processes include the assembly and association of genetic material samples with associated diseases, the preparation of microarrays with representative genetic material samples in a pattern best suited for analysis as well as manipulation, and delivery of assimilated and compiled data across a computer network. Various aspects of these embodiments are discussed in FIGS. 1-4 below.

FIG. 1 shows a diagram of an overall method 100 for preparing a blood sample to be used in a broad-based disease association gene transcript test according to an embodiment of an invention disclosed herein. The method may include drawing a blood sample from a patient scheduled for genotyping in step 110. Of course, in order to assimilate a broad-based set of data across several diseases, blood samples are typically drawn from several sources. It should be noted that any tissue suitable for gaining access to genetic material (e.g., DNA and/or RNA) may be used, such as liver tissue. Blood cells are easily collected and easily transported making this source for DNA/RNA efficient and effective. The blood sample may typically be collected using a suitable blood collection device such as blood collection tubes that are available from Paxgene™.

In medical practice, venipuncture or venepuncture (also known as phlebotomy, blood draw, drawing blood or taking blood) is the process of obtaining a sample of venous blood. Typically, a 5 ml to 25 ml sample of blood is adequate for a sample from a single person, but the exact required amount will typically depend on what the particular purpose for the blood test is. For the purposes of the tests disclosed herein, 5 ml is sufficient, and, in effect, far less quantities may typically be adequate. In many circumstances, the collection of a blood sample may be done by a phlebotomist, although nurses, doctors and other medical staff are also trained to take blood.

Blood is most commonly obtained from the median cubital vein, on the anterior forearm (the side opposite the elbow). This vein lies close to the surface of the skin, and there is not a large nerve supply, making the location best suited for this purpose. Once obtained, the blood sample may be moved from the collection device into a storage container for later use and analysis.

The sample is typically properly tagged and labeled by an anonymous yet traceable patient identification. That is, all measures are taken to comply with the Health Insurance Portability and Accountability Act (HIPAA) such that the blood sample is identifiable but also protected from accidental disclosure of privileged information. At the time of collection, additional demographic information may be stored (e.g., written on a tag, stored in a computer database) with the blood sample. Such demographic information may include a number of different patient characteristics and descriptions, such as age, sex, country of origin, race, specific health issues, occupation, birthplace, current living location, etc.

Within the context of the method detailed in FIG. 1, step 110, the collection of the blood sample, may be accomplished at any location at any facility. The remaining steps of FIG. 1 need not occur in any proximity to step 110. In fact, the collection of blood samples may be accomplished simultaneously in multiple locations and then all properly tagged blood samples may be transported (e.g., shipped) to a processing center wherein one or more of the remaining steps of the method of FIG. 1 may be accomplished.

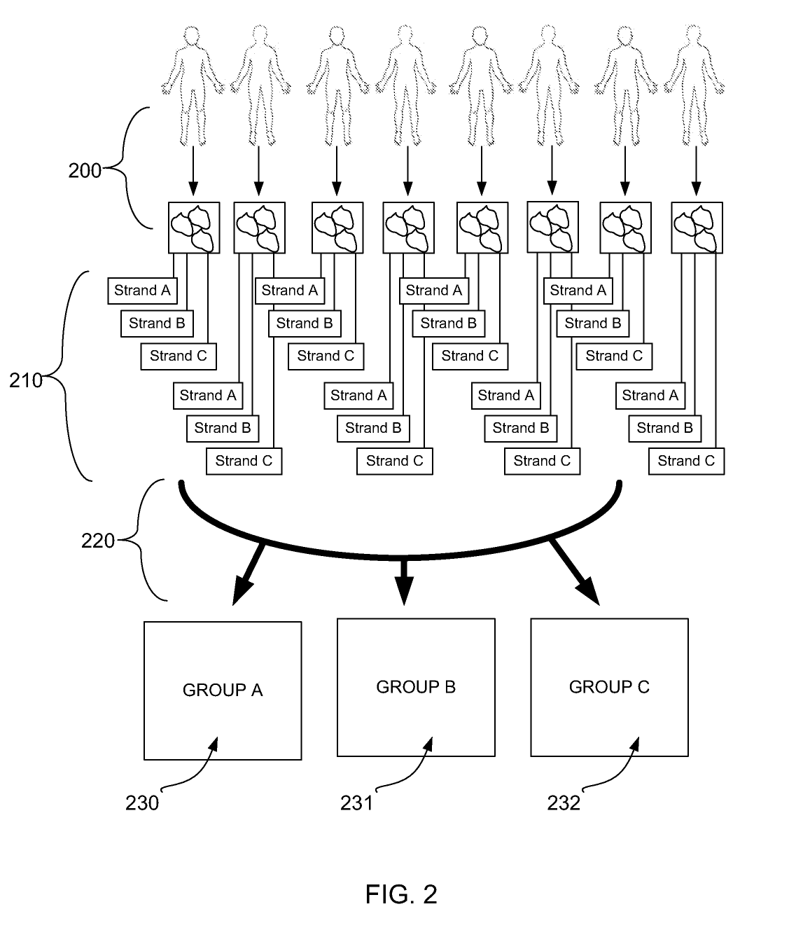

Specific genetic material, such as RNA from the blood sample, may then be detected and isolated in step 112 using an RNA isolation kit such as those that are available from Qiagen™. As mentioned above, RNA isolation may be accomplished at the same physical location as collection or may be accomplished at a remote laboratory after collection. The genetic material isolation process is described in more detail below with respect to FIG. 2.

At step 114, specific sequences in an RNA sample may be amplified using a fluorescence process that may be specific to pre-determined strands of RNA such as available from Illumina™ in a product entitled DASL™. In an alternative embodiment, specific sequences in DNA may also be amplified using a similar fluorescence process that may be specific to pre-determined strands of DNA such as available from Illumina™ in a product entitled Golden Gate™.

The isolation of genetic materials is typically followed by amplification of fluorescently labeled copies that may then be hybridized to specific probes attached to a common substrate, i.e., a microarray. At step 116, the isolated and amplified samples of genetic material may be grouped according to identified sets of strands of genetic material. The groups may be arranged in a specific pattern in bead pools on a microarray according to a predetermined format. Such predetermined formats may include a standard format suitable for individual analysis of all identified genes in isolated RNA/DNA strands. Other predetermined formats may include a side-by-side comparison to one or more control groups of similar genes from control group samples. Other formats may include specific sets of genes suitable for broad-based disease association, multiple sclerosis association, broad-based diagnostics collection, broad-based predictive treatment data sets, or any other association of genes with samples. Once the microarray has been created in a specific pattern, the emergence of patterns and the like may be ready for analysis at step 118. The preparation of each microarray is described in more detail in U.S. patent application Ser. No. ______ entitled, “Method and System for Preparing a Microarray for a Disease Association Gene Transcript Test,” assigned to IGD-Intel of Seattle, Wash. The formats for arranging samples in a microarray typically follow specifics associated with the groupings of blood samples as discussed below with respect to FIG. 2.

FIG. 2 shows a diagrammatic representation of a method for collecting blood samples from several sources and identifying strands of genetic material for grouping according to an embodiment of an invention disclosed herein. In an overview of one method disclosed herein, one may begin the method by collecting a plurality of similar blood samples from a plurality of similar sources, the blood samples suitable for genetic code isolation and analysis. Then, identifiable strands of genetic material in each blood sample may be detected and isolated such that the strands of genetic material identifiable by a gene sequence or nucleotide sequence.

Next, for each blood sample, as an identifiable strand emerges, the samples may be separated into sets of samples with similar identifiable strands and then each set of isolated strand samples of genetic materials may be then grouped into groups of genetic material from each of the plurality of blood samples, such that each group comprises similar identifiable strands of genetic material from each blood sample. Once grouped, each group of genetic material may be associated with a disease relevant to the identifiable strands comprising each group or any other relevant data that may be useful for diagnostics. Aspects of these broad-based steps are discussed below.

In FIG. 2, several different sources of genetic material may typically be used to obtain several different samples of genetic material. This step is represented in the aggregate at step 200 in FIG. 2 and may be associated with the individual step 110 of FIG. 1. As a result, several different and identifiable samples of genetic material may then be processed to detect and isolate specific genetic material for assimilation into an aggregate context. One such process includes RNA isolation.

RNA isolation comprises obtaining high quality, intact RNA and is often the most critical step in performing many fundamental molecular biology experiments, including the various broad-based disease association gene transcript tests disclosed herein and disclosed in related patent application assigned to IGD-Intel To assure success, the RNA isolation procedure typically includes some additional steps both before and after the actual RNA purification. As such, treatment and handling of tissue or cells prior to RNA isolation and storage of the isolated RNA are also important aspects of obtaining the best RNA yield from genetic material samples. Finding the most appropriate method of cell or tissue disruption for a specific starting material is important for maximizing the yield and quality of an RNA preparation.

The process of RNA isolation, as used herein, refers to a process of detecting and highlighting (i.e., hybridizing) specific strands of RNA that may be present in any given sample. It is well understood that in any “isolated” RNA sample, there typically exists a very large number of identifiable strands of genetic sequence. By applying known methods for hybridization, such as combining complementary, single-stranded nucleic acids into a single molecule, nucleotides will bind to their complement under normal conditions. Thus, in a process called annealing, two complementary strands will bind to each other readily. However, due to the different molecular geometries of the nucleotides, a single inconsistency between the two strands will make binding between them more energetically unfavorable. Measuring the effects of base incompatibility by quantifying the rate at which two strands anneal can provide information as to the similarity in base sequence between the two strands being annealed. As a result, fluorescents highlighting may be then used to identify the existence of a specific strand of RNA within the sample.

Specific gene sequences (i.e., nucleotide sequences) may be identified when detecting and isolating strands of genetic material from each sample at step 210. On an aggregate level, each sample may typically have a first strand, such as STRAND A, such that all gene sequences that may be identified as STRAND A may be isolated and the sample separated from all other strands. Likewise, STRAND B for each sample may be also isolated and its respective sample separated. The case is also the same for STRAND C and every other identifiable strand of genetic material in each sample. Although, only 3 specific strands are shown in FIG. 2, it is well understood in the art that the potential strands that may be isolated number in the thousands. At the time this application is filed, at least 1142 specific and identifiable strands are available for detection and isolation in each sample. As but three examples, STRANDS A, B, and C may typically be identified by the gene sequences as follows:

STRAND A: (Typically Associated with Disease A)

AATCAGAAGAGATGATTCAACTTGGTGACCAAGAAGTTTCTGAGCTTTGT
GGCCTACCAACGGAGAAACTGGCTGCAGCAGAGCGAGTACTTCGTTCCAA
CATGGACATCCTGAAGCCAA

STRAND B: (Typically Associated with Disease B)

AATCAGAAGAGATGATTCAACTTGGTGACCAAGAAGTTTCTGAGCTTTGT
GGCCTACCAATGGAGAAACTGGCTGCAGCAGAGCGAGTACTTCGTTCCAA
CATGGACATCCTGAAGCCAA

STRAND C: (Typically Associated with Disease C)

GGGACCATAGGCCGGTCTGGTGTCCGTGTGGAGGGCCCCCTGAAGATCCG
AGGCCAGGTGGTGGATGTGGAACAGGGGATCATCTGCGAGAACATCCCCA
TCGTCACGCCCTCAGGAGAG

Such isolation processes may comprise the isolating of genetic material based on strands of RNA as identified by a specific gene sequence as described above. Additionally, the isolation of genetic material may be based upon a gene sequence associated with a gene expression indicative of a disease, a gene sequence associated with a gene expression indicative of a trait, a gene sequence associated with a gene expression indicative of a phenotype, and/or a gene sequence associated with a gene expression indicative of a genotype.

With all strands detected, isolated, and identified, each set of strands (i.e., all samples with STRAND A isolations) across all samples may be grouped together for additional association and analysis at step 220. As such, all expressions of STRAND A may be grouped into GROUP A 230, all expressions of STRAND B may be grouped into GROUP B 231 and all expressions of STRAND C may be grouped into GROUP C 232. Such grouping allows for the assimilation of data on an aggregate level based on various gene expressions as compared to a number of aggregate level aspects of assimilated data. Specifically, demographic information about the source of a sample may be associated with each sample.

Additionally, aggregating information associated with each blood sample may be accomplished through the groupings of similar strands. Such aggregating includes associating a blood sample exhibiting an expression of a gene sequence indicative of a first disease with the demographic information about the blood sample, associating a blood sample exhibiting an expression of a gene sequence indicative of a first disease with another blood sample exhibiting an expression of a gene sequence indicative of the first disease, associating a blood sample exhibiting an expression of a gene sequence indicative of a first disease with a blood sample exhibiting an expression of a gene sequence indicative of a second disease, associating a blood sample exhibiting an expression of a gene sequence indicative of a first disease with a treatment associated with the first disease, and associating a blood sample exhibiting an expression of a gene sequence indicative of a first disease with a specific polymorphism.

With any number of associations in place from the groupings, statistical data from the aggregated blood samples based on associations of one blood sample with another may be extrapolated. Such statistical data may include expression rates, inter-related expression rates, etc.

In one embodiment, each set of samples of isolated strands may be arranged into bead pools prior to grouping the sets and then each bead pools may be arranged in respective groups on a glass microarray. Arranging bead pools on a microarray provides an industry-standard format for analysis and databasing. Glass microarray analysis experiments typically require between 5 ng and 5 μg of total RNA per slide for sample labeling and hybridization. Thus, microarray-based gene expression analysis of very small samples, tissue biopsies, or other clinical samples is difficult due to the very low amounts of total RNA recovered from the samples. Linear amplification of RNA from small samples produces sufficient quantities of RNA for sample labeling and hybridization. Since the amplification technique is highly reproducible and maintains representation of the gene expression in the original sample, it is typical for probe synthesis offered by most manufacturers of commercially available microarrays.

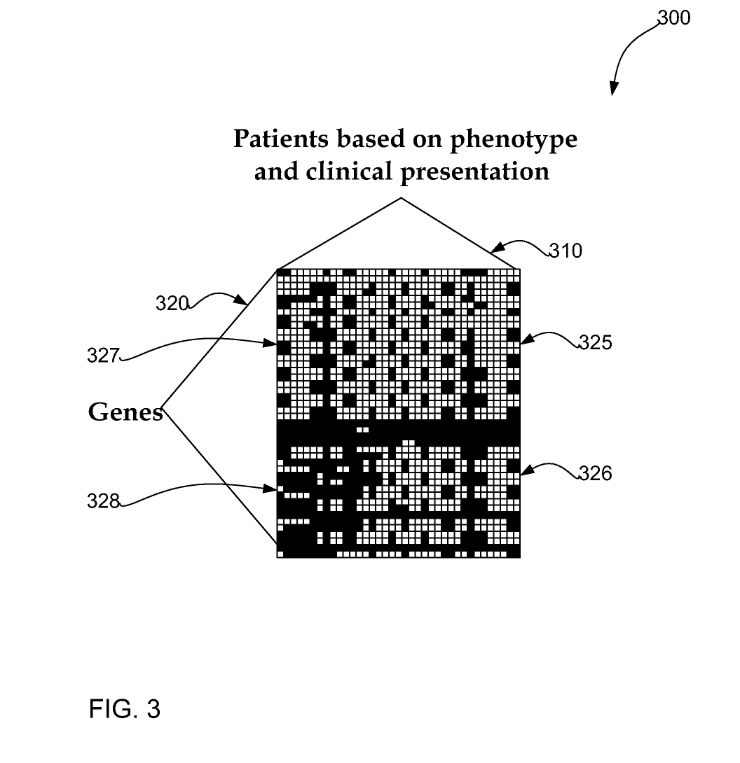

FIG. 3 shows a typical arrangement of data 300 extrapolated from blood samples in a microarray suitable for analysis in a broad-based disease association gene transcript test according to an embodiment of an invention disclosed herein. In this embodiment, the data 300 is characterized by a graphical arrangement of different identified genes 320 in the horizontal axis and patient data according to phenotype in the vertical axis 310. In this example data arrangement, data from 50 patients and data from about 50 genes are plotted for analysis. Several other embodiments exists as specific patterns of the presence of phenotypes or lack thereof determine the type of information to be garnered from each prepared microarray. As a result of this embodiment, specific patterns emerge indicating a likelihood of occurrence of an SNP and/or levels of expression transcript. This is exemplified in region 326 and 327. Likewise other regions may show the likelihood of a lack of occurrence of an SNP, such as is exemplified in region 325 and 328.

With such a data arrangement 300 available for analysis and coupled with multiple additional prepared arrays, broad-based data about the occurrence or absence of diseases and/or specific gene sequences begins to emerge. The displayed data arrangement 300 may be scanned and intensity data extracted to associate presence/absence of genetic material exhibiting specific levels of gene expression in the original sample. Such a process may typically be referred to an RNA expression analysis and red and green patterns emerge as a standard manner of data interpretation in the industry. This data may be assimilated in a large database of information together with additional information such as diagnosis and treatment information, to provide a multitude of information about a large number of data sets. As the data is assimilated, a comprehensive literature search offering substantiated associations of disease with gene sequence alterations may be provided. The data are rendered anonymous and uploaded into a central repository that allows cross-sample comparison and ultimately, earlier detection of disease.

Application of this unique set of probes will offer a low cost genomic assessment of an individual's state of health through a new and useful clinical diagnostic. Additionally, adding or deleting probes that relate to a given disease, as new information presents in the literature may further enhance the benefits of the clinical diagnostic. Adding probe content as information expands is an additional course of action, as will be appreciated by others in the art. Further yet, the clinical diagnostic may be expanded such that components may be tested as separate, and/or all inclusive tests that address different diseases or lifestyle concerns. For the purposes of this disclosure, however, the microarray will not be discussed further.

Information that may now be gleaned from the groupings of sets of genetic material may be aggregated into in a computer readable medium accessible by a server computer, e.g., a database. Then, such data may be accessed by any connected client computer such that information is provided from the aggregated data to a client computer upon a request from the client computer to the server computer.

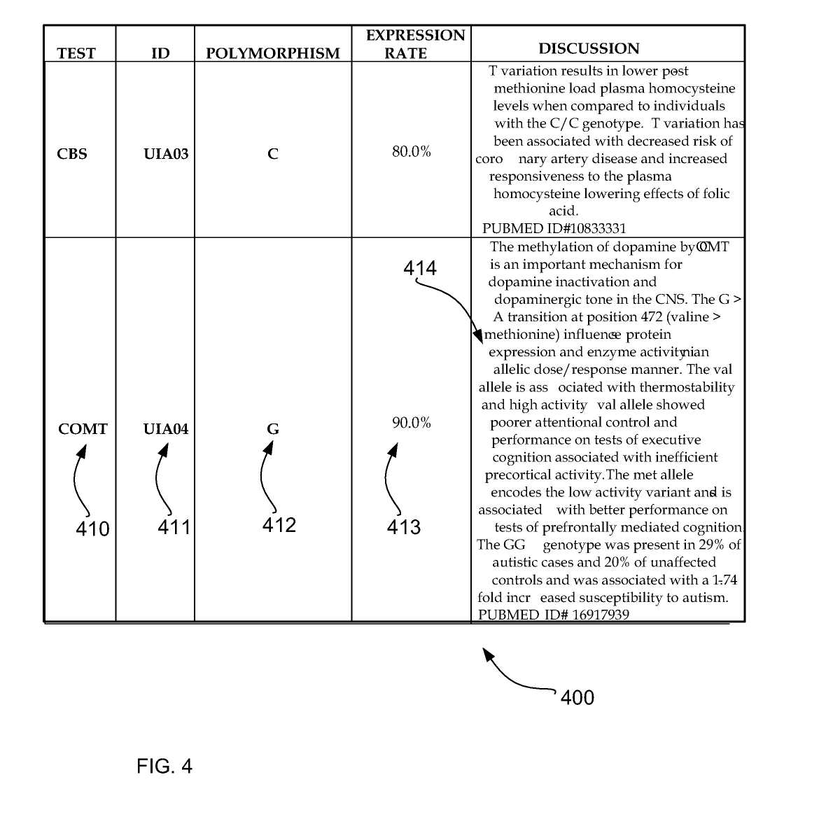

The data associated with the groupings of genetic material may be arranged in a data structure 400 according to FIG. 4. In FIG. 4, the data structure may associate a specific test 410, an ID 411, a polymorphism 412, an expression rate 413, and a discussion 414. With such a database of broad-based disease association data, doctors may more readily and easily compare individual gene expression results against aggregate data. Additionally, the aggregate data will be reinforced by the inclusion of the doctor's patient's data. Further yet, through associative analysis of the broad-based data in the database, predictive and diagnostic data may emerge.

A specific combination of nucleotide sequences taken from isolated regions of the human genome may be reflected as custom content on a platform independent gene expression microarray. A complete list of nucleic acid sequences form the elements analyzed within this human genome examination may form the basic nature of a gene transcript test, which is intended for clinical use in effectively detecting transcribed alterations in the genetic code that have a documented relationship with disease, association with therapeutic response, and/or treatment for disease. The content of the test may assess RNA through quantitative (measurement and assessment of transcript present within the tissue) and qualitative (measurement of genomic regions) means.

This microarray may comprise probe sequences isolated to detect regions within a given gene that most effectively indicate expression levels and that represent polymorphic sections indicating which sequence from the genome an individual is actually expressing. The nucleotide sequences deemed present in the amplified genetic material isolated from standard blood draw and/or disease affected tissue, may be detected by hybridizing the amplified genetic material to the array and analyzing a hybridization pattern resulting from the hybridization.

Association of test results with documentations and testimonials of clinical relevance may be assimilated and documented as conclusions formed through a comprehensive compilation of peer-reviewed literature are assessed. Ongoing modifications to these documented cases may be performed through quarterly protocol assessment and maintenance of a peer-to-peer physician support network supported through existing and impending corporate associations.

In a system embodiment of the method described above, a system for preparing a blood sample for a broad-based disease association gene transcript test may be realized as well. The system may include one or more blood collection devices operable to collect a plurality of similar blood samples from a plurality of similar sources, the blood samples suitable for genetic material isolation and analysis. Additionally, the system includes a genetic material isolation device operable to isolate identifiable strands of genetic material in each blood sample, the strands of genetic material identifiable by a unique gene sequence. Further yet, the system may include an identification apparatus operable to separating each identifiable strand into sets of similar identifiable strands for each blood sample.

Additional sub-systems, such computing devices and the like may also be included in the aggregate system. These computing devices typically include a grouping apparatus operable to group each set of isolated strands of genetic materials into groups of genetic material from each of the plurality of blood samples, such that each group comprises similar identifiable strands of genetic material from each blood sample and an association device operable to associate each group of genetic material with a disease relevant to the identifiable strands comprising each group.

Further yet, additional devices and apparatuses may include an amplification device operable to amplify a strand of genetic material for isolation, a data assimilation server computer operable to store information about the groupings of genetic material, various apparatuses associated with preparing and manipulating bead pools and microarrays as well as viewing devices for viewing a microarray.

Paper reporting of the test results may indicate the outcome from a subset of 1 to 50 genetic sequences. Additional reporting for the remaining analyzed sequences may be made available through alternative measures. These measures may enable physicians to access their patient's information relative to all other patients having ordered the test through a variety of associative clustering methods (hierarchical, divisive, and associative). The concept of creating real-time genotype/phenotype association accessible to physician-to-physician networks may be further promoted as a useful goal. Physicians will be able to analyze their own patient's data relative to all other data existing individuals who have had the test performed.

The polymorphisms assessed may be single nucleotide polymorphisms (SNPs), deletions, and/or deletion-insertion sequences. Further, the polymorphisms predicted to be present in the amplified genetic material may already be determined. Further yet, the nucleic acid sample may be genomic DNA, cDNA, cRNA, RNA, total RNA or mRNA. With these variations, the SNP, deletion, or insertion may be associated with a disease, the efficacy of a drug, and/or associated with predisposition towards/against development of aforementioned ailment(s). Typically, output data may be packaged in a CD and delivered to a customer, such as a subscribing physician.

While the subject matter discussed herein is susceptible to various modifications and alternative constructions, certain illustrated embodiments thereof are shown in the drawings and have been described above in detail. It should be understood, however, that there is no intention to limit the claims to the specific forms disclosed, but on the contrary, the intention is to cover all modifications, alternative constructions, and equivalents falling within the spirit and scope of the claims.

What is claimed is:

1. A method for preparing a plurality of blood samples for a broad-based disease association gene transcript test, the method comprising:
collecting a plurality of similar blood samples from a plurality of similar sources, the blood samples suitable for genetic material analysis;
detecting and isolating identifiable strands of genetic material in each blood sample, the strands of genetic material identifiable by a unique gene sequence;
for each blood sample, separating each identifiable strand into sets of similar identifiable strands;
grouping each set of isolated strands of genetic materials into groups of genetic material from each of the plurality of blood samples, such that each group comprises similar identifiable strands of genetic material from each blood sample; and
associating each group of genetic material with a disease relevant to the identifiable strands comprising each group.

2. The method of claim 1, further comprising amplifying each blood sample using a fluorescence process specific to the isolated genetic material.

3. The method of claim 1; further comprising:
arranging the sets of genetic material into bead pools prior to grouping the sets; and
laying out the bead pools on an array, such that the bead pools are arranged into their respective groups.

4. The method of claim 1 wherein the isolating of genetic material comprising isolating strands of RNA as identified by a gene sequence.

5. The method of claim 4 wherein the isolated strands of RNA comprise a gene sequence associated with a gene expression indicative of a disease.

6. The method of claim 4 wherein the isolated strands of RNA comprise a gene sequence associated with a gene expression indicative of a trait.

7. The method of claim 4 wherein the isolated strands of RNA comprise a gene sequence associated with a gene expression indicative of a phenotype.

8. The method of claim 4 wherein the isolated strands of RNA comprise a gene sequence associated with a gene expression indicative of a genotype.

9. The method of claim 1 wherein the isolating of genetic material comprising isolating strands of DNA as identified by a gene sequence.

10. The method of claim 9 wherein the isolated strands of DNA comprise a gene sequence associated with a gene expression indicative of one of a group comprising a disease, a trait, a genotype, and a phenotyope.

11. The method of claim 1; further comprising associating demographic information about the source of a sample with each sample.

12. The method of claim 11, further comprising aggregating information associated with each blood sample, the aggregating comprising:
associating a blood sample exhibiting an expression of a gene sequence indicative of a first disease with the demographic information about the blood sample;
associating a blood sample exhibiting an expression of a gene sequence indicative of a first disease with another blood sample exhibiting an expression of a gene sequence indicative of the first disease;
associating a blood sample exhibiting an expression of a gene sequence indicative of a first disease with a blood sample exhibiting an expression of a gene sequence indicative of a second disease;
associating a blood sample exhibiting an expression of a gene sequence indicative of a first disease with a treatment indicative of the first disease; and
associating a blood sample exhibiting an expression of a gene sequence indicative of a first disease with a specific polymorphism.

13. The method of claim 12; further comprising extrapolating statistical data from the aggregated blood samples based on associations of one blood sample with another.

14. The method of claim 12 further comprising:
storing the aggregated data in a computer readable medium accessible by a server computer; and
providing information from the aggregated data to a client computer upon a request from the client computer to the server computer.

15. The method of claim 1, wherein the associated disease comprises a disease from a group of diseases including: Addison's disease, anemia, asthma, atherosclerosis, autism, breast cancer, estrogen metabolism, Grave's disease, hormone replacement therapy, major histocompatibility complex (MHC) genes, infectious disease screening panel, longevity, lupus, multiple sclerosis, obesity, osteoarthritis, prostate cancer, type 2 diabetes and Multiple Sclerosis.

16. A system for preparing a blood sample for a broad-based disease association gene transcript test, the system comprising:
one or more blood collection devices operable to collect a plurality of similar blood samples from a plurality of similar sources, the blood samples suitable for genetic material isolation and analysis;
a genetic material detection and isolation device operable to isolate identifiable strands of genetic material in each blood sample, the strands of genetic material identifiable by a unique gene sequence;
an identification apparatus operable to separating each identifiable strand into sets of similar identifiable strands for each blood sample;
a grouping apparatus operable to group each set of isolated strands of genetic materials into groups of genetic material from each of the plurality of blood samples, such that each group comprises similar identifiable strands of genetic material from each blood sample;
and an association device operable to associate each group of genetic material with a disease relevant to the identifiable strands comprising each group.

17. The system of claim 16, further comprising an amplification device operable to amplify a specified strand of genetic material.

18. The system of claim 16, further comprising a data assimilation server computer operable to store information about the groupings of genetic material.

19. The system of claim 16, further comprising a bead pool apparatus operable to assimilate and manipulate the samples of genetic material into a plurality of bead pools.

20. The system of claim 19, further comprising:
an assembly device operable to assemble the bead pools onto a microarray; and
an viewing device operable to view the assembled microarray.
